(19) United States
(12) Patent Application Publication
(10) Pub. No.: US 2015/0366999 A1
(43) Pub. Date: Dec. 24, 2015

(54) Nanomagnetic modification of angiography contrast dye and a novel process for its removal using intravascular catheter based electromagnet system.

(71) Applicants: Amod Amritphale, Las Vegas, NV (US); Nupur Amritphale, Las Vegas, NV (US); Chowdhury H Ahsan, Las Vegas, NV (US); Frank Smart, New Orleans, LA (US)

(72) Inventors: Amod Amritphale, Las Vegas, NV (US); Nupur Amritphale, Las Vegas, NV (US); Chowdhury H Ahsan, Las Vegas, NV (US); Frank Smart, New Orleans, LA (US)

(21) Appl. No.: 14/311,333

(22) Filed: Jun. 22, 2014

(51) Int. Cl. A61K 49/04; A61M 5/00; A61M 25/09; A61B 19/00; A61N 2/02; A61B 6/02; A61M 25/00; A61B 5/00; A61K 49/22; A61N 2/00

(52) U.S. Cl. CPC A61K 49/0438; A61K 49/04; A61K 49/225; A61M 5/007; A61M 25/09; A61N 2/002; A61N 2/02; A61B 6/02; A61M 25/0012; A61B 5/6853; A61B 5/6851; A61B 19/5244; A61B 2019/524

(57) ABSTRACT

Angiographic dye is used in multiple diagnostic and therapeutic procedures. These procedures have a very high accuracy of diagnostic and therapeutic abilities but have potential nephrotoxicity. This patent relates to (i) an improvement in angiography therapy technology using a Nanomagnetic modification of angiography contrast dye and (ii) a novel process for its removal using intravascular catheter based electromagnet system. This patent also relates to in-laboratory and/or in-house modification of the existing radiocontrast dye using various methods for making it amenable to manipulation and/or extraction off the blood stream, by the catheter based electro magnet system as described in the present invention. This patent also relates to the process of reno-protection from potentially nephrotoxic contrast dye by its electromagnetic removal and its use thereof.

### TECHNICAL FIELD

This patent relates to a) an improvement in angiography therapy technology using a Nanomagnetic modification of angiography contrast dye and b) a novel process for its removal using intravascular catheter based electromagnet system. This patent also relates to in-laboratory and/or in-house modification of the existing radiocontrast dye using various methods for making it amenable to manipulation and/or extraction off the blood stream, by the catheter based electromagnet system as described in the present invention. This patent also relates to the process of reno-protection from potentially nephrotoxic contrast dye by its electromagnetic removal and its use thereof.

### BACKGROUND

Angiographic dye is used in multiple procedures—diagnostic and therapeutic procedures especially IR guided and coronary procedures, and CT scans, dye based sonograms/echo and other vascular procedures. All these procedures used the conventional low molecular weight iodine containing dye for visualization which has potential nephrotoxicity. These procedures have a very high accuracy of diagnostic and therapeutic abilities but have potential nephrotoxicity and the ability to delineate the disease procedures better than non-contrast procedures. These dyes have to pass through the kidney for getting excreted and therefore is responsible for undesirable nephrotoxicity in patients with normal or abnormal kidney function. Preferred Intravascular X-ray contrast agents possess a combination of desirable properties such as maximum x-ray opacity; biological safety; high water solubility; chemical stability; low osmolality; and low viscosity.

As a prior art, research has focused mainly on decreasing high osmolality as it is correlated with undesirable physiologic adverse reactions to x-ray contrast media, e.g., nausea, vomiting, heat and pain. As a prior art there are numerous guidelines, modalities and treatment procedures for reno-protection including “hydration” and use of “sodium bicarbonate” before the procedure or going for Hemodialysis/Peritoneal Dialysis (HD/PD) after the procedure for early removal of the dye from the system. As a precautionary measure no two dye-requiring procedures are done within 24 hours of each other until emergently required, so that the patients' body has some time to recuperate from the nephrotoxic insult.

These aforementioned disadvantages of using the conventional iodine containing dye/contrast media leads to:

1. Higher nephrotoxicity
2. Inability to perform multiple contrast requirement procedures within 24 hrs until emergently required.
3. Advancement of kidney diseases secondary to osmotic load.
4. Need for Hemodialysis/Peritoneal Dialysis (HD/PD) to remove the dye from the system to prevent nephrotoxicity.
Historically (as a prior art) there have been numerous researches aiming at reducing the osmolality and viscosity of the contrast dye and which are as follows:
1. There is an extensive literature on X-ray contrast media development; e.g., G. Brooke Hoey, “Synthesis and Biological testing of Non-ionic Iodinated X-ray contrast media” 1980 Investigative Radiology Vol. 15 and U. Speck, “Contrast Agents: X-ray Contrast Agents and Molecular Imaging—A Contradiction?” Handbook of Experimental Pharmacology 1853; 167-174.
2. A significant advancement in the area of triiodobenzene based X-ray contrast media has been the development of non-ionic triiodobenzoic acid derivatives such as iopamidol, iohexol and ioversol. In general, aqueous solutions of these non-ionic agents have less osmolality than previous agents and hence, provide greater patient comfort when injected. Adverse reactions, especially in the sensation of pain, warmth, and hemodynamic effects are greatly reduced as compared to the ionic triiodobenzoic acid derivatives. However, at equal iodine concentrations, the viscosity values of these non-ionic formulations are higher than for formulations of ionic triiodobenzoic acid based contrast agents.
3. Further reduction of osmolality of X-ray contrast media resulted from the introduction of non-ionic dimeric agents such as iotrolan and iodixanol. These agents because of isoosmolality values, as compared to the non-ionic monomeric agents, provide even greater patient comfort by reducing nausea and vomiting upon intravenous injection and by causing much less pain upon peripheral arterial injection. However, the frequency and intensity of delayed side reactions in patients are higher for such non-ionic dimers. The viscosity of such non-ionic dimeric agent-based formulations is also substantially greater than for the corresponding monomeric analogs. Thus there remains a need for safe formulations of X-ray contrast media with low viscosity and low osmolality.
4. In U.S. Pat. No. 5,698,739, Sovak describes a separate class of dimeric non-ionic X-ray contrast media with at least one primary carboxamide group as a substituent, an example of this class of dimers being Iosmin. According to Sovak, the presence of primary carboxamide group conferred higher iodine content and sterically exposed the hydrophobic character of the neighboring iodine molecules ensuring the formation of aggregates and thus lowering the osmolality.
5. Another attempt to optimize formulations involves combining a monomer with low viscosity value with a dimer of low osmolality value wherein the substituents on the iodinated aromatic groups are similar. U.S. Pat. No. 5,695,742, discloses injectable aqueous compositions comprising mixtures of non-ionic iodinated aromatic monomers and non-ionic iodinated aromatic dimers having an intermediate osmolality value compared to the pure solutions wherein the mixtures are also disclosed as having a lower viscosity than expected.
6. With the purpose of decreasing the delayed side reactions seen with the non-ionic dimers, U.S. Pat. No. 8,679,460 B2 discloses mixtures comprising monomers and dimers of ionic and non-ionic triiodoaromatic compounds as well as gadolinium complex compounds. Since the mixtures include an ionic contrast agent, the osmolality of such a mixture would be higher than the osmolality value for the pure nonionic dimer contrast agent.
7. In U.S. Pat. No. 8,679,460 B2, Muthunadar P. Periasamy, Brian D. Doty discovered novel monomer-dimer mixtures having improved osmolality and viscosity profiles in which the dimer has at least one primary carboxamide group in the triiodobenzene nuclei and the monomer has no primary carboxamide groups in the triiodobenzene nuclei.
   Even after the aforementioned research advances the angiography contrast agent still remains potentially nephrotoxic with a cumulative dose effect.

### ADVANTAGE OVER THE PRIOR ART

1. A novel Process of making a new hybrid organo-inorganic precursor containing iodine/radiopaque containing contrast dye
2. A novel Process to remove the novel dye of the present invention using catheter based intravascular electromagnet system to reduce undesirable nephrotoxicity.
3. A novel Process to modify the conventionally used dye in-house prior to administration to the patient that makes it amenable for the point 2 above by modifying it into a nano-magnetic radiocontrast agent which under the influence of the catheter based electromagnet gets adhered to the high surface area of biocompatible polymer outer surface of the electromagnet of the present invention.
4. This invention is particularly important as it helps to prevent the undesirable nephrotoxicity of the contrast agent by its early removal and/or extraction from the bloodstream using magnetic properties. This helps protect the kidney tissue from bearing the insult of the contrast agent during its excretion from the body.

### SUMMARY

In one aspect this invention proposes a novel angiographic (iodine or other radiopaque dye) which can be removed from the system using the novel electromagnetic apparatus of the present invention.

In another aspect this invention proposes a method of Primary/secondary modification of the conventional contrast agents before administration to the patient, thereby converting the conventional contrast agent to a new hybrid molecule which has a property similar to the novel contrast agent of the present invention.

In another aspect this invention proposes a novel intravascular based catheter based electromagnet system which has a core of miniature electromagnet, intermediate catheter tubing and an outer high surface area biocompatible polymer surface for trapping the said contrast media of the present invention.

In another aspect this invention proposes a novel nephroprotective method and its use thereof wherein any contrast agent can be modified to a nano-magnetic agent by primary or secondary modifications.

### DESCRIPTION OF DRAWINGS

FIG. 1 is a schematic view of the nanoparticle of the present invention with its various layers, shell and attachments.

FIG. 2 is a schematic view of the nano-core of the present invention with its direct interaction with the commercially available contrast molecules.

FIG. 3 is the intravascular catheter/guidewire based miniature electromagnet system with its multiple layers for the process of early extraction of contrast agent of the present invention using procedure dependent continuous or pulsed electromagnetic field application.

FIG. 4 is the catheter/para-catheter system housing the miniature electromagnet of the present invention.

FIG. 5 is the external controller setup system integrated with the miniature electromagnet. The aforementioned external controller setup for the miniature electromagnet can be used to produce continuous or pulsed magnetic field of desired duration using the controller using intravenous access.

FIG. 6 describes the intra-arterial placement of the miniature electromagnet system as desired either above or below the level of renal arteries.

### DETAILED DESCRIPTION

Angiographic dye is used in multiple procedures—diagnostic and therapeutic procedures especially IR guided and coronary procedures, and CT scans, dye based sonograms/echocardiograms and other vascular procedures. All these procedures used the conventional low molecular weight iodine containing dye for visualization which has undesirable nephrotoxicity. As a prior art research has focused on decreasing high osmolality as it is correlated with undesirable physiologic adverse reactions to x-ray contrast media, e.g., nausea, vomiting, heat and pain. As a prior art there are numerous guidelines, modalities and treatment procedures for reno-protection including “hydration” and use of “sodium bicarbonate” before the procedure or going for Hemodialysis/peritoneal dialysis (HD/PD) after the procedure for early removal of the dye from the system. This invention is a major advancement in angiography agent technology wherein the agent is magnetically removed and/or extracted from the bloodstream even before it interacts with the kidney tissue for excretion. This is the basis of its potential nephroprotection.

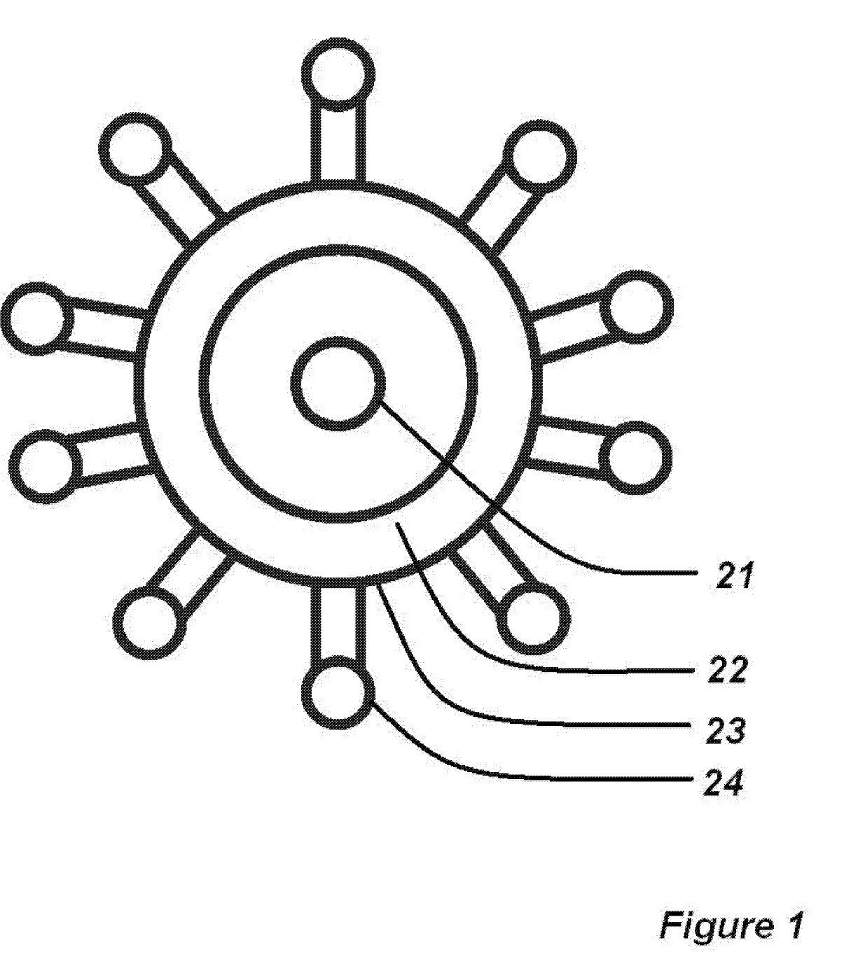

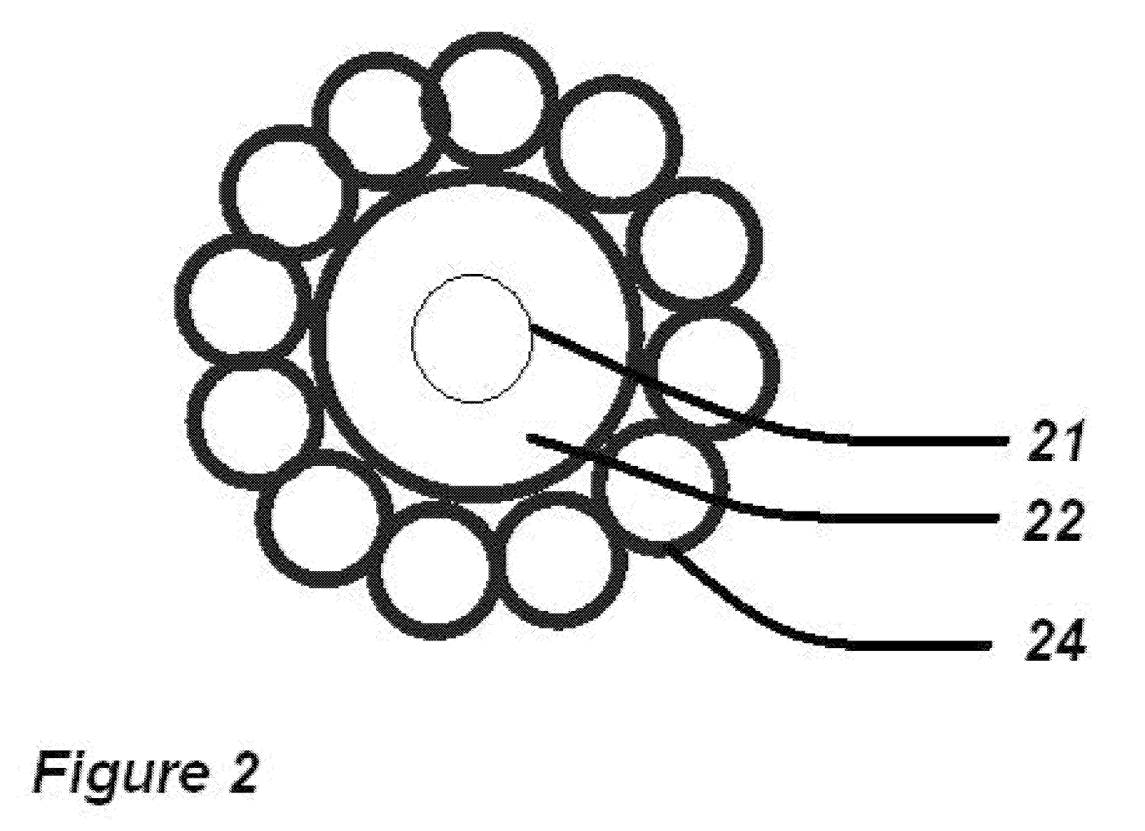

The novel angiographic dye hereby used interchangeably with the term agent and or contrast is a composite organo-inorganic hybrid precursor moiety with a nanomagnetic core with the shell made of one or more of iomeral, optiray or other available agent molecules. Referring to FIG. 1, Nanoparticle core 21 used in the present invention can be purchased from Alfa Aesar or synthesized de novo as described by Zeng et al. (US 20100047180 A1). The physical characteristics of the magnetic nanoparticles and the effect of heating have also been described by Zeng et al. (US 20100047180 A1). The nanoparticles are novel advanced magnetic nanoparticle with organo-inorganic hybrid precursor moieties with novel surface interface 22 compatible for both organic as well as inorganic species and thus enabling synergistic features from the functionality of the nanoparticles core with the shell 23 and or attachment 24 made of the existing contrast agents iomeral, optiray or other available agent molecules and the therapeutic agents, which can be introduced in another coating nano-core. Referring to FIG. 2, the nanoparticle core 21 along with its novel surface interface 22 compatible for both organic as well as inorganic spec of the present invention can have direct affinity to the attachments 24 without intervening shell 23. The nanomagnetic particles are “organo-inorganic” hybrid precursor derived nanoparticles instead forming them by individual system or a portion thereof. This hybrid precursor derived nanomaterials will further increase and expand the desired functionality spectrum and thus enabling the newer application spectrum of the angiography agents secondary to the novel surface-nanotechnology of the hybrid organo-inorganic interactive surface 22, 23 and its enhanced functionality thereof.

In the present patent application, the synthesis of core 21 of the said agent which is in-turn made of hybrid nano size materials of cobalt ferrite involves a novel approach of making homogenous solution of iron glycolato as well as cobalt glycolato, simultaneously using their respective acetate compounds in desired stoichiometric ratio in the presence of ammoniated ethylhydrazine and the hydrolysis was carried out by designing two systems i.e. firstly adding hybrid homogenous solution drop-wise into the ammoniated ethylhydrazine solution and secondly reversing the adding of ammoniated solution to mix solution of iron and cobalt glycolato species in a synergistic reaction. These two synergistic reaction approaches are responsible for providing desired polymeric hybrid iron cobalt species in nano size materials in imparting the desired functionality for the intended application. The nanoparticles of the present invention can also be prepared using the aforementioned synergistic reaction modification of the method described by Satyajit V. Shukla, et al in US Pat. Publication 2013/0105397; Al Satoshi Nakamura et al (2006) J. Mater. Res., 21: 5:1336-41 and Bernd Wicklein and German Salazar-Alvarez (J. Mater. Chem. A, 2013, 1, 5469-78). The particles can be chosen from gold-coated cobalt particles (Co@Au), silver-coated cobalt particles (Co@Ag), gold-coated magnetic iron oxide (Fe₃O₄@Au), silver-coated magnetic iron oxide (Fe₃O₄@Ag) and gold-coated cobalt/iron mixtures (CoFe@Au), iron platinum alloys (FePt), or a combination thereof. Gold- or silver-coated cobalt particles (Co@Au or Co@Ag) are typically used. Fabrication of Co@Au particles is described in Lu et al. (2005) Langmuir 21(5):2042-50. Magnetite containing magnetic particles having a gold or a silver shell are discussed in Madhuri, M. et al. (2005) Journal of Colloidal and Interface Science 286:187-194. The said nanomaterial core has nana-surface properties which makes it a perfect substrate for adsorbing the commercially available dye including but not limited to either iomeral, optiray or other agents.

In embodiments, the size of the particles and their composition depends on alloys in the nana-core of the agent with their outer coating 22, 23 or as an attachment 24 comprising of iomersal or optiray. In another embodiment, the nanoparticles core can have at least one dimension (e.g., the thickness for a nanoplate, the diameter for a nanosphere, a nanocylinder and a nanotube) that is less than 600 nm, e.g., less than 100 nm. In particular embodiments, the magnetic particles have a spherical shape with a diameter ranging from about 1 nm to 120 nm; more typically, from about 5 nm to 60 nm; from about 4 nm to 32 nm; from about 5 to 15 nm; or about 12 nm. The novel advanced magnetic nanoparticle with organo-inorganic hybrid precursor moieties facilitate the surface 23 of the nano-core 21 to become compatible for both organic as well as inorganic species 24 and thus enabling synergistic features from the functionality of the nanoparticles core and their interaction with angiography dye of the prior art and other therapeutic agents. Magnetic particles are further discussed by Jon Dobson in Drug Development Research 67:55-60 (2006). In one embodiment, ferromagnetic FeCo particles are used (Hutten, A. et al. (2005) Journal of Magnetism and Magnetic Materials 293:93-101). Such particles typically range in size from about 1 to 11 nm and are superparamagnetic. The ioversal used as the shell 23/attachment 24 of the nano-core 21 of the present invention can be prepared as described in the U.S. Pat. No. 6,596,904 B1. The mode of delivery and mixing properties remain the same as described in the U.S. Pat. No. 6,596,904 B 1.

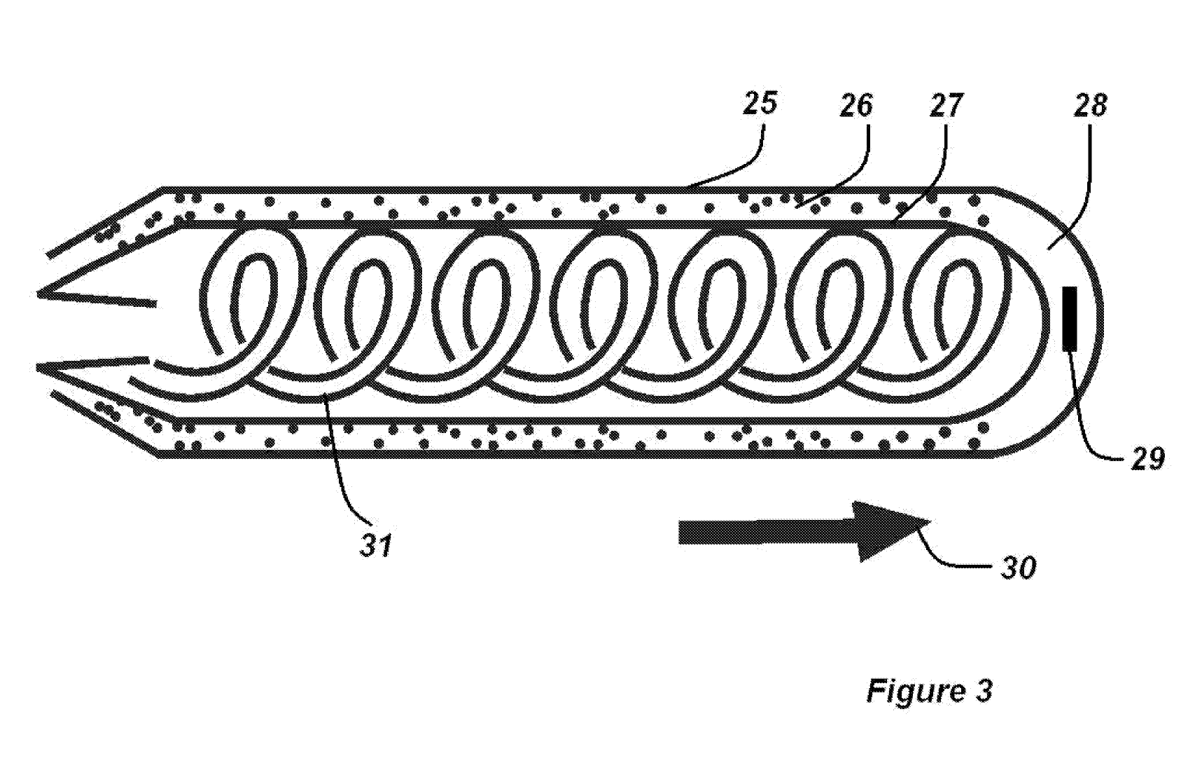

In another embodiment, referring to FIG. 3 the miniature electromagnet is housed within the innermost core of body of the para-catheter or the guidewire. The outer most layer with the adsorbing capacity 25 is structured to hold a large amount of angiography dye nanoparticles of the present invention. The miniature electromagnet of the present invention has an outer coating 26 of the biocompatible catheter material. The second coating is electrically inert polymer 27 which covers in the miniature electromagnet electrical coil and apparatus 31. The advancing end 28 of the electromagnet system of the present invention houses a locus identifier system 29 which helps to guide the fluoroscopic advancement of the catheter system in the desired direction 30. The catheter and/or its coatings can be obtained commercially from Abbott Laboratories Vascular Enterprises Limited or prepared de novo as described in U.S. Pat. No. 8,323,432 B2. The second layer of the catheter or guidewire housing the miniature electromagnet of the present invention can be obtained commercially from Abbott Laboratories Vascular Enterprises Limited or can be made de novo using the method described by U.S. Pat. No. 4,323,071A. The second layer 26 is permanently fixed to the innermost layer 27 which in turn houses the miniature electromagnet 25 of the present invention.

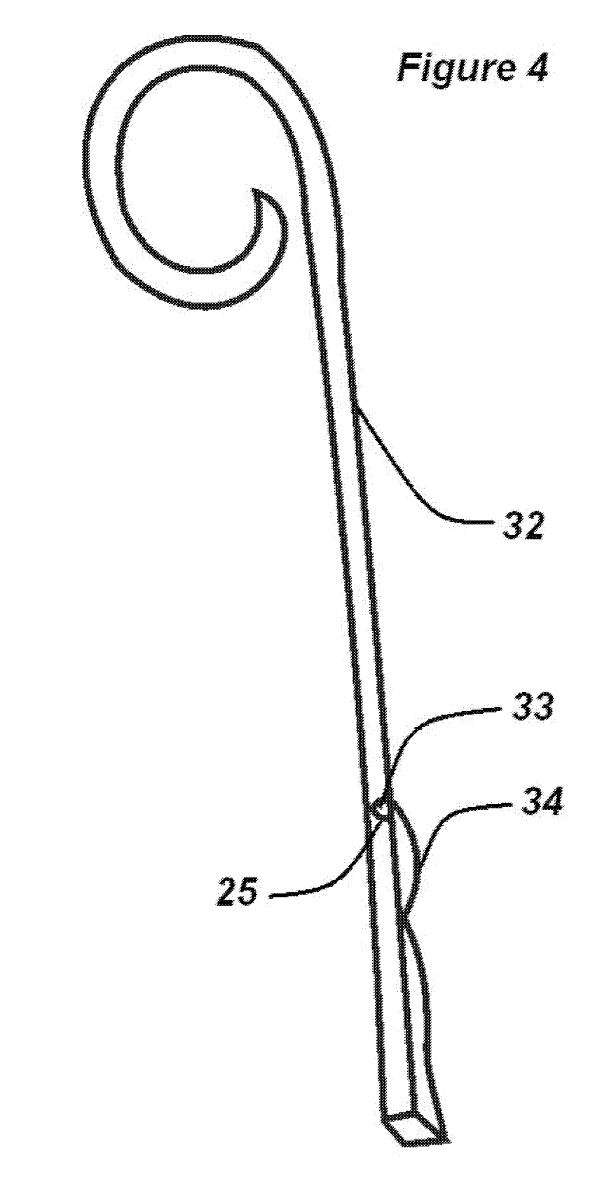

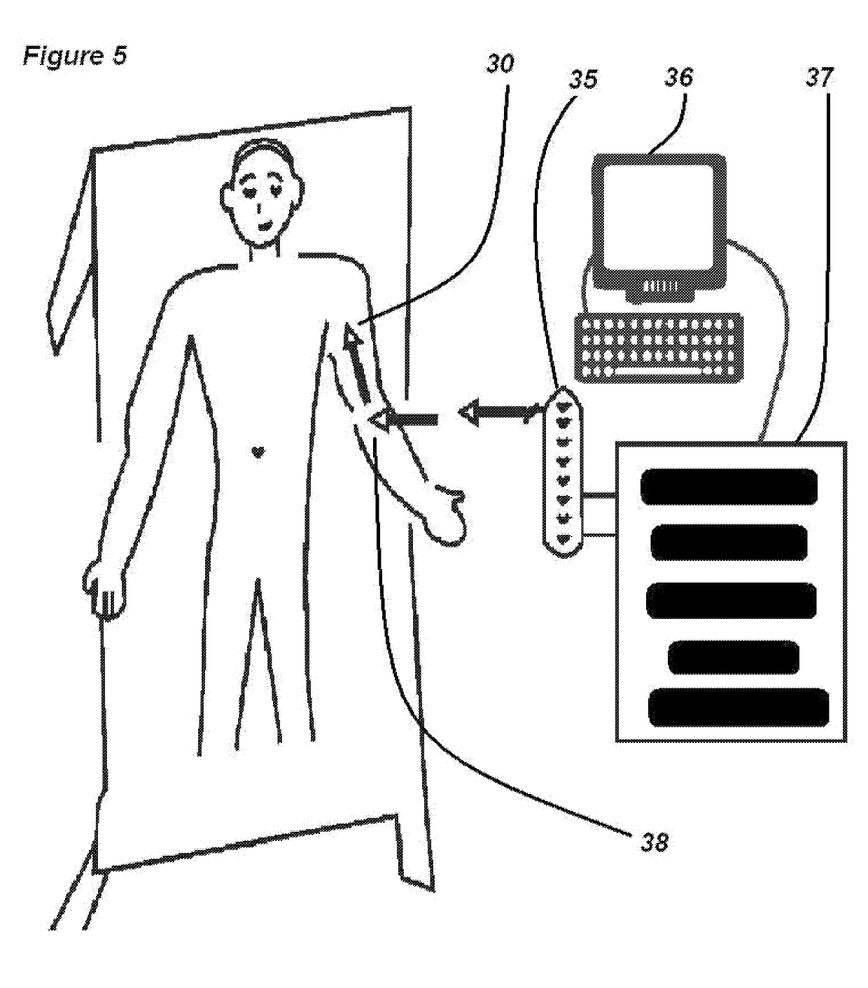

Referring to FIG. 4, the miniature electromagnet of the present invention is housed within the innermost core of body of the catheter or the guidewire 33. It can also be housed in a para-catheter 34 which can be rolled over all commercially available catheter systems. The miniature electromagnet can be obtained commercially from Aurora by Northern Digital Inc. or can be manufactured de novo using the process described by U.S. Pat. No. 5,191,306 A and U.S. Pat. No. 6,498,477 B1. Referring to FIG. 5, the external controller setup 36 and 37 and software system 36 comes integrated with the commercially obtained miniature electromagnet by Aurora by Northern Digital Inc. or can be manufactured de novo as described in U.S. Pat. No. 6,498,477 B1. The aforementioned external controller setup for the miniature electromagnet can be used to produce continuous or pulsed magnetic field of desired duration using the controller 35.

In another embodiment the strength of the magnetic field can be modulated as per the requirement in individual subjects using the apparatus 35 as described in U.S. Pat. No. 6,498,477 B1. The exact location of the electromagnet inside of the catheter can be controlled using the locus identifier 29 and envisioned using the process described by US 20120215094 A1 for advancing in the desired direction 30.

In another embodiment the third or the outermost layer 25 can be fixed to the inner layer or removable and replaceable during the duration of use. This layer 25 has a high surface area nanostructured organic biopolymer coating that can be prepared de novo using Vapor-Phase Fascile method as described by Goyal, Swati et al in Nanotechnology, IEEE Transactions; Vol 9, Issue: 5; 618-624. This layer can be removed and replaced with a fresh third layer during any procedure involving contrast agent if it gets saturated with the adsorbed agent under the effect of the in-housed miniature electromagnet. The saturation is seen as the decrease in ability of the catheter of the present invention to adsorb at least one percent of the contrast agent after at least 30 seconds of pulsed or continuous magnetic field application.

In embodiments the novel angiographic dye hereby used interchangeably with the term agent and or contrast is a composite organo-inorganic hybrid precursor moiety with a naomagnetic core with the shell made of one or more of iomeral, optiray or other available agent molecules can be used as a replacement of the conventionally used iomeral, optiray or other available agents without the need of any different storage conditions, control or applicator apparatus, mixing abilities mixing and interaction with the blood.

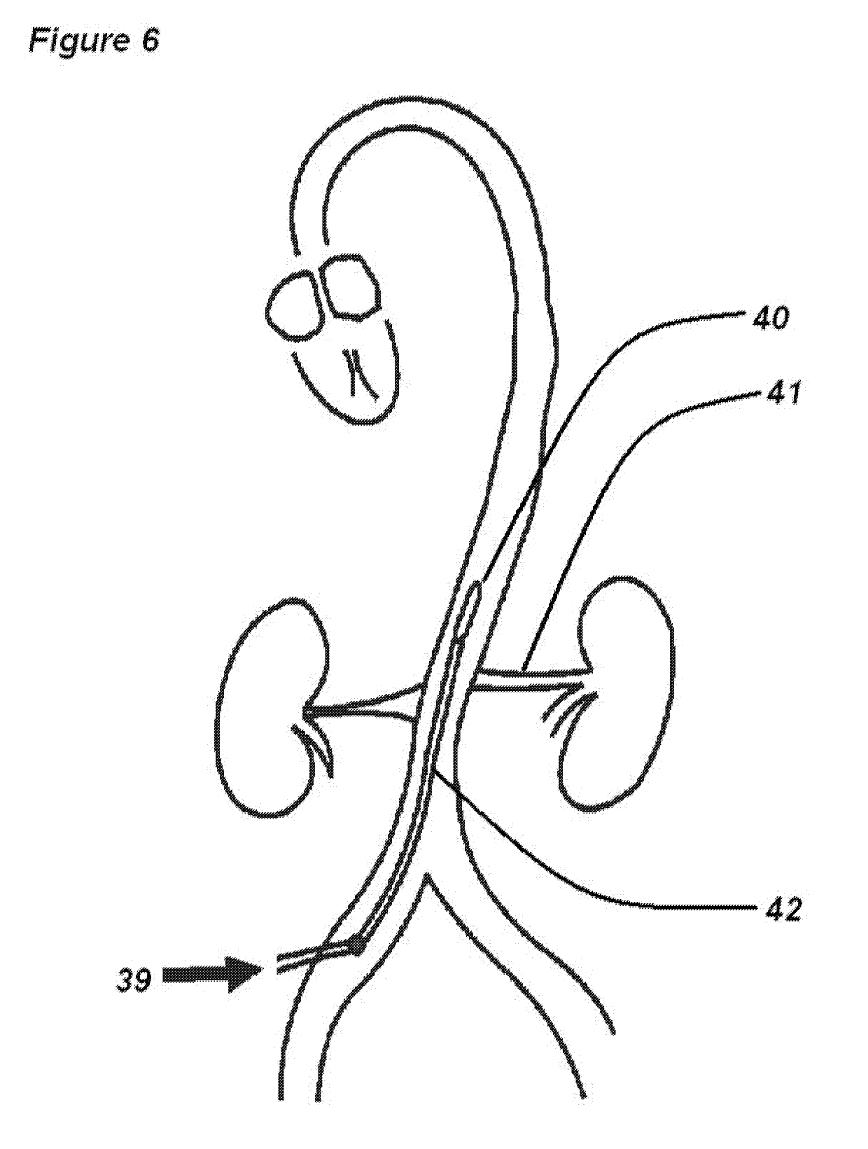

Referring to FIG. 6 the intravascular based miniature electromagnet system of the present invention can be used either intravenous access 38 and/or intra-arterial access 39 depending on the requirement of the procedures and patient specific characteristics. In preferred conditions, the intravascular based miniature electromagnet system 40 of the present invention will be placed intra-arterial 39 in case of the diagnostic and/or therapeutic coronary intervention procedures requiring contrast agents and intravenously 38 in case of diagnostic and/or therapeutic scans in which the dye is administered intravenously. The miniature electromagnetic system can be advanced as desired to keep its locus above or below the level of the Renal arteries 41. This is accomplished by advancing the catheter/paracatheter/guidewire 42 during the invasive procedure.

In another embodiment, the intravascular based miniature electromagnet system of the present invention can generate continuous or pulsatile magnetic field of desired strength depending on the procedural requirement or limitation. In preferred conditions, the miniature electromagnet system will be used to produce pulsatile magnetic field to prevent any interaction or distortion of images produced during the coronary and/or other vascular interventions, while a continuous magnetic field will be preferably used during the extraction phase of diagnostic and/or therapeutic scans using contrast agents.

In another embodiment, the intravascular based miniature electromagnet system of the present invention leads to reduction in the amount of contrast agent excreted through the kidney and is preferentially extracted by the highly adsorbing surface of the biocompatible polymer coating of the outermost layer of the catheter of the present invention. This thereby reduces the nephrotoxicity as a dose cumulative effect as described in the present invention.

The complete disclosure of the patent documents, patents and publications cited in the background and elsewhere herein are incorporated by reference in their entirety as if each were individually incorporated. Illustrative embodiments of this invention and figures are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof. Other embodiments are within the scope of the following claims.

What is claimed is:

1. A novel aqueous injectable composition hereby called as contrast agent or angiography dye or novel iodine contrast, useful to obtain images during X-ray examinations, CT scans and vascular and coronary interventions, comprising, dissolved into an aqueous medium, a composite organo-inorganic hybrid precursor moiety with a nanomagnetic core with the shell made of one or more of iomeral, optiray or other available agent molecules.

2. The composition according to claim 1 wherein the novel agent has a core of composite organo-inorganic hybrid precursor moiety with the shell and or attachments made of one or more of iomeral, optiray or other available agent molecules.

3. The composition according to claim 1 wherein the agent is amenable to modulation and/or extraction using intravascular magnetic field application.

4. The composition according to claim 1 wherein there is decreased nephrotoxicity secondary to early extraction of the said agent under intravascular magnetic field.

5. A novel intravascular catheter/guidewire based miniature electromagnet system for the process of early extraction of contrast agent of the present invention using procedure dependent continuous or pulsed electromagnetic field application.

6. A novel intravascular catheter/guidewire based miniature electromagnet system of the claim 5 wherein the applied magnetic field can be externally controlled in order to selectively increase or decrease the rate of adsorption of the contrast agent of the present invention to facilitate smooth extraction from the intravascular system.

7. A novel intravascular catheter/guidewire based miniature electromagnet system of the claim 5 wherein the applied magnetic field is preferable pulsatile when used with coronary and/or other vascular interventions to prevent any interaction of or distortion of images produced during the procedure, while a continuous magnetic field is preferred during the extraction phase of diagnostic and/or therapeutic scans using contrast agents.

8. A novel intravascular catheter/guidewire based miniature electromagnet system of the claim 5 wherein the catheter based system can be introduced intra-arterially and/or intravenously depending on the requirement of the procedures and patient specific characteristics. In preferred conditions, the intravascular based miniature electromagnet system is placed intra-arterial in case of the diagnostic and/or therapeutic coronary intervention procedures requiring contrast agents and intravenously in case of diagnostic and/or therapeutic scans in which the agent is administered intravenously.

9. A novel catheter/paracatheter or guidewire based miniature electromagnet housing system with multiple concentric and/or adjuvant tubular spaces for use during the diagnostic and/or therapeutic intravascular and/or coronary interventions, and computed tomography based procedures.

10. A novel catheter/paracatheter or guidewire based miniature electromagnet housing system of claim 9 wherein, the miniature electromagnet of the present invention can be housed in the core tube of the catheter or the adjacent guidewire.

11. A novel catheter/paracatheter or guidewire based miniature electromagnet housing system of claim 9 wherein the second layer from within is a biocompatible fixed layer to prevent any electrical transmittance to the body fluids.

12. A novel catheter/paracatheter or guidewire based miniature electromagnet housing system of claim 9 wherein the outermost layer is composed of high surface area nanostructured organic biopolymer coating prepared de novo using Vapor-Phase Fascile method. This coating is hereby used for obligatory adsorption of the agent of the present invention under the effect of the electromagnetic field.

13. A novel catheter/paracatheter or guidewire based miniature electromagnet housing system of claim 9 wherein the outermost layer is removable and replaceable depending upon the amount of administered agent load to effectively extract the agent of the present invention thereby reducing the cumulative dose dependent nephrotoxicity.
